(19) United States
(12) Patent Application Publication
(10) Pub. No.: US 2021/0322386 A1
(43) Pub. Date: Oct. 21, 2021

(54) METHODS OF DELAYING AND REVERSING ALZHEIMER'S DISEASE PROGRESSION

(71) Applicant: Loma Linda University, Loma Linda, CA (US)

(72) Inventors: Salvador SORIANO, Loma Linda, CA (US); Dong Kyu SHIN, Loma Linda, CA (US); Karina MAYAGOITIA, Loma Linda, CA (US)

(73) Assignee: Loma Linda University, Loma Linda, CA (US)

(21) Appl. No.: 17/250,185

(22) PCT Filed: Jun. 10, 2019
(86) PCT No.: PCT/US2019/036395

§ 371 Date: Dec. 10, 2020

Related U.S. Application Data
(60) Provisional application No. 62/683,587, filed on Jun. 11, 2018.

(51) Int. Cl. A61K 31/4196; A61K 45/06; A61P 25/28

(52) U.S. Cl. CPC A61K 31/4196; A61P 25/28; A61K 45/06

(57) ABSTRACT

In one aspect, methods of reversing or delaying the progression of Alzheimer's disease in a subject having Alzheimer's disease or delaying the onset of Alzheimer's disease in a subject having mild cognitive impairment are provided. In some embodiments, the method comprises administering to the subject an oxysterol inhibitor.

### CROSS-REFERENCE TO RELATED APPLICATION

This application claims priority to U.S. Provisional Application No. 62/683,587, filed Jun. 11, 2018, the disclosure of which is hereby incorporated by reference in its entirety for all purposes.

### BACKGROUND OF THE INVENTION

Alzheimer's disease is a progressive brain disorder characterized by memory loss, impaired cognition, and impaired reasoning or judgment. Alzheimer's disease is the most common form of dementia, and it is estimated that about 5 million Americans may have the disease.

Despite years of research, the mechanisms that lead to Alzheimer's disease pathology remain unknown. The amyloid cascade hypothesis proposes that Alzheimer's disease is caused by the accumulation, oligomerization, and aggregation of amyloid-beta peptide (Aβ) in extracellular deposits. Aβ is proteolytically derived from the Amyloid Precursor Protein (APP), and therefore, therapeutic approaches to the treatment of Alzheimer's disease have focused on preventing the accumulation of Aβ in the brain in order to ameliorate or halt the disease. However, numerous drugs aimed at reducing the burden of Aβ in the brain have failed to treat Alzheimer's disease. See, e.g., Castello et al., BMC Neurology, 2014, 14:169. Moreover, a significant portion of the cognitively healthy population show accumulation of AP in the brain (see, e.g., Aizenstein et al., Arch Neurol, 2008, 65:1509-1517), indicating that Aβ is neither necessary nor sufficient to initiate the disease.

Mild cognitive impairment (MCI), also known as incipient dementia and isolated memory impairment, is a neurological disorder that generally occurs in adults aged over 60, and involves noticeable cognitive impairments beyond normal aging, which minimally disrupt daily activities. While some instances of MCI remain stable with time, or resolve, it is estimated that around 30% of individuals diagnosed with MCI develop Alzheimer's disease within three years (see, Mitchell and Shiri-Feshki, Acta Psychiatrica Scandinavica, 2009, 119:252-265). Of the MCI subtypes, patients with amnestic MCI (a-MCI) appear to be at risk for developing Alzheimer's disease. To date, there are no approved drugs for the treatment of MCI.

Accordingly, there remains a need for methods of treating Alzheimer's disease and preventing or delaying the onset of Alzheimer's disease in subjects having Mild Cognitive Impairment (MCI).

### BRIEF SUMMARY OF THE INVENTION

In one aspect, methods of delaying or reversing progression of Alzheimer's disease in a subject are provided. In some embodiments, the method comprises:
administering to a subject having Alzheimer's disease an oxysterol inhibitor.

In some embodiments, the subject has mild Alzheimer's disease. In some embodiments, the subject has moderate Alzheimer's disease. In some embodiments, the subject has severe Alzheimer's disease. In some embodiments, the subject has early-onset Alzheimer's disease.

In some embodiments, the oxysterol inhibitor is a 27OHC inhibitor. In some embodiments, the 27OHC inhibitor inhibits the production of 27-hydroxycholesterol by the enzyme CYP27A1.

In some embodiments, the oxysterol inhibitor is an aromatase inhibitor.

In some embodiments, the oxysterol inhibitor is selected from anastrozole, exemestane, letrozole, vorozole, formestane, testolactone, aminoglutethimide, 1,4,6-androstatrien-3,17-dione, 4-androstene-3,6,17-triune, bicalutamide, posaconazole, and derivatives or combinations thereof. In some embodiments, the oxysterol inhibitor is anastrozole.

In some embodiments, administration of the oxysterol inhibitor to the subject delays progression of mild Alzheimer's disease to moderate Alzheimer's disease.

In some embodiments, administration of the oxysterol inhibitor to the subject delays progression to severe Alzheimer's disease.

In some embodiments, administration of the oxysterol inhibitor to the subject reverses the progression of Alzheimer's disease in the subject.

In some embodiments, treatment of a subject with the oxysterol inhibitor results in an increased level of one or more biomarkers (e.g., RTKN2) in the subject.

In another aspect, methods of delaying the onset of Alzheimer's disease in a subject having Mild Cognitive Impairment (MCI) are provided. In some embodiments, the method comprises:
administering to the subject having MCI an oxysterol inhibitor.

In some embodiments, the subject has amnestic MCI.

In some embodiments, the oxysterol inhibitor inhibits the production of 27OHC by the enzyme CYP27A1.

In some embodiments, the oxysterol inhibitor is an aromatase inhibitor.

In some embodiments, the oxysterol inhibitor is selected from anastrozole, exemestane, letrozole, vorozole, formestane, testolactone, aminoglutethimide, 1,4,6-androstatrien-3,17-dione, 4-androstene-3,6,17-triune, bicalutamide, posaconazole, fadrozole, dexmedetomidine, ravuconazole, and derivatives or combinations thereof. In some embodiments, the oxysterol inhibitor is anastrozole.

In some embodiments, treatment of a subject with the oxysterol inhibitor results in an increased level of expression of one or more biomarkers (e.g., RTKN2) in a sample from the subject.

In another aspect, the disclosure provides methods of preventing and/or delaying the onset of Alzheimer's disease and/or Mild Cognitive Impairment in a subject who is at least 60 years old and/or has a cholesterol level of 200 mg/dL or above by administering to the subject an oxysterol inhibitor.

In some embodiments of this aspect, the oxysterol inhibitor is a 27OHC inhibitor. In some embodiments, the 27OHC inhibitor inhibits the production of 27-hydroxycholesterol by the enzyme CYP27A1.

In some embodiments of this aspect, the oxysterol inhibitor is an aromatase inhibitor.

In some embodiments of this aspect, the oxysterol inhibitor is selected from the group consisting of anastrozole, exemestane, letrozole, vorozole, formestane, testolactone, aminoglutethimide, 1,4,6-androstatrien-3,17-dione, 4-androstene-3,6,17-trione, bicalutamide, posaconazole, fadrozole, dexmedetomidine, ravuconazole, and derivatives or combinations thereof. In particular embodiments, the oxysterol inhibitor is anastrozole.

In some embodiments of this aspect, administration of the oxysterol inhibitor to the subject results in an increased level of expression of rhotekin 2 (RTKN2) in the subject.

### BRIEF DESCRIPTION OF THE DRAWINGS

FIG. 1 is a schematic showing conversion of cholesterol to 27-hydroxycholesterol (27OHC) by the CYP27A1 enzyme.

FIG. 2 shows that anastrozole ameliorates the negative effect of a high-cholesterol diet on cognitive function in mice as measured by Y-maze test.

### DETAILED DESCRIPTION OF THE INVENTION

### I. INTRODUCTION

Disclosed herein are methods of delaying or reversing the progression of Alzheimer's disease and methods of delaying the onset of Alzheimer's disease. It has been postulated that in the development of Alzheimer's disease, amyloid-beta peptide (Aβ) is a protective molecule that is regulated in response to chronic stress in the brain, such as oxidative stress, metabolism dysregulation (e.g., cholesterol homeostasis and insulin resistance), genetic factors, and inflammation response. According to this hypothesis, called the adaptive response hypothesis, the presence of Aβ is evidence of an ongoing stress process, rather than a marker of disease initiation. See, e.g., Castello et al., BMC Neurol, 2014, 14:169; and Castello et al., Ageing Research Reviews, 2014, 13:10-12.

It has been recently described that Amyloid Precursor Protein (APP) regulates an adaptive cytoprotective response to an early marker of cholesterol dysregulation in the Alzheimer's disease brain and protects the brain from cholesterol oxidation. See, International Application No. PCT/US2017/065367, titled “Methods of Diagnosing Alzheimer's Disease and Risk of Progression to Alzheimer's Disease,” filed Dec. 8, 2017, incorporated by reference herein. The oxysterol 27-hydroxycholesterol (27OHC) is an early marker of cholesterol dysregulation in the late-onset Alzheimer's disease brain. See, Marwarha et al., J Alzheimers Dis 19, 1007-1019 (2010); Prasanthi et al., PLoS One 6, e26420 (2011). in vivo, hydroxycholesterols such as 27OHC are formed through enzymatic conversion of cholesterol or by free radical autoxidation. Without being bound to a particular theory, it is postulated that oxysterol accumulation as a result of dysregulated cholesterol metabolism and failed APP protection against this oxysterol stress trigger the inflammatory and apoptotic pathways of Alzheimer's disease, leading to progressive neurodegeneration. Enzymes that catalyze the oxidation of cholesterol include enzymes in the cytochrome P450 superfamily. For example, the production of 27-hydroxycholesterol (27OHC) is associated with the enzyme CYP27A1.

Thus, in one aspect, the present disclosure relates to halting the onset or progression of Alzheimer's disease by inhibiting the production of oxysterols, such as 27OHC, that are pathogenic effectors of Alzheimer's disease. Without being bound to a particular theory, it is believed that by limiting the pool of cholesterol molecules available for conversion to oxysterols or preventing the pool of cholesterol molecules from being converted to oxysterols (e.g., by inhibiting the enzymes that catalyze this conversion, such as CYP27A1), the progression of Alzheimer's disease can be delayed or reversed in a subject having Alzheimer's' disease or the onset of Alzheimer's disease in a subject having mild cognitive impairment (MCI) can be delayed.

### II. DEFINITIONS

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about.” The term “about” refers to (+) or (−) 10%, e.g., (+) or (−) 5%, of a stated value.

The singular forms “a,” “an,” and “the” include plural referents unless the context clearly dictates otherwise. Thus, for example, reference to “a compound” includes a plurality of compounds.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

As used herein, “Alzheimer's disease” refers to a disease characterized by progressive cognitive impairment. The symptoms of Alzheimer's disease typically worsen over time as the disease progresses, with the disease typically progressing through three stages: “mild” (an early-stage form of Alzheimer's disease), “moderate” (a middle-stage form), and “severe” (a late-stage form). In mild Alzheimer's disease, symptoms may include, for example, memory loss, losing or misplacing objects, trouble remembering names or recalling words, increased difficulty with planning or organizing, taking longer to complete normal daily tasks, and repeating questions. In moderate Alzheimer's disease, which is typically the longest stage of the disease for many patients, damage occurs in areas of the brain that control language, reasoning, sensory processing, and conscious thought. In this stage, symptoms may include, for example, forgetfulness of events or of one's one personal history, problems recognizing family and friends, inability to learn new information, difficulty carrying out multi-step tasks, impulsive behavior, changes in sleep patterns, hallucinations, delusions, and paranoia. In severe Alzheimer's disease, memory and cognitive skills continue to worsen, patients typically lose the ability to respond to their environment, carry on a conversation, and/or control movement, and patients require a high level of assistance with daily activities and personal care.

In some embodiments, a patient has “late onset” Alzheimer's disease, which refers to a form of Alzheimer's disease in which the patient exhibits clinical symptoms of the disease after about age 65. In some embodiments, a patient has “early onset” Alzheimer's disease, which refers to a form of Alzheimer's disease in which a patient exhibits the onset of clinical symptoms of the disease prior to the age of 65. In some embodiments, patients having early onset Alzheimer's disease exhibit the onset of clinical symptoms of the disease in their 30s, 40s, or 50s. In some embodiments, the early onset Alzheimer's disease is early onset familial Alzheimer's disease (FAD), which is a hereditary form of Alzheimer's disease caused by autosomal dominant mutations that affect APP processing.

As used herein, “Mild Cognitive Impairment” refers to a disorder that is characterized by a decline in cognitive abilities (such as memory and thinking skills) that is greater than expected for an individual's age and education level but that does not interfere notably with activities of daily life. See, Gauthier et al., Lancet, 2006, 367:1262-70. In some embodiments, a subject has amnestic-MCI (a-MCI).

Clinical tests for diagnosing Alzheimer's disease and Mild Cognitive Impairment are described in the art. See, e.g., Boustani et al., Screening for Dementia, Systematic Evidence Reviews, No. 20, 2003.

As used herein, an “oxysterol inhibitor” refers to a compound that inhibits the production of an oxysterol, an oxygenated derivative of cholesterol, or that inhibits the activity of an oxysterol. In some embodiments, an oxysterol inhibitor inhibits the production of an oxysterol from cholesterol by blocking the activity of an enzyme, such as a cytochrome P450 oxidase, that catalyzes the production of an oxysterol from cholesterol.

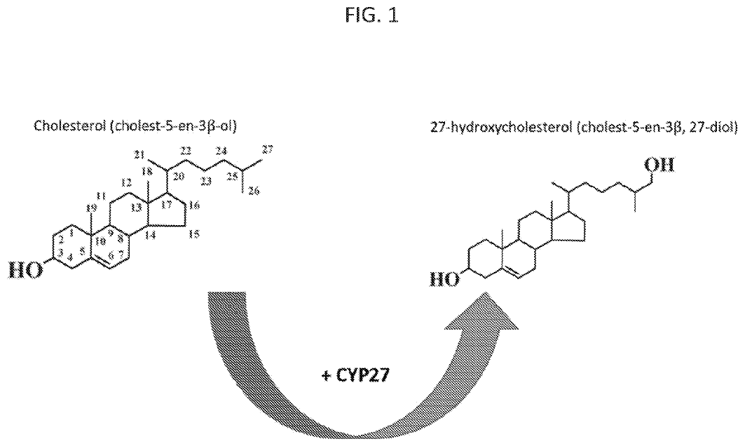

As used herein, a “27OHC inhibitor” refers to a compound that inhibits the production of 27-hydroxycholesterol (27OHC) from cholesterol or inhibits the activity of 27OHC. In some embodiments, a 27OHC inhibitor inhibits the production of 27OHC from cholesterol by blocking the activity of the CYP27A1 enzyme. “CYP27A1” refers to cytochrome P450 family 27 subfamily A member 1, a gene that encodes cytochrome P450 oxidase, also known as sterol 27-hydroxylase. Sterol 27-hydroxylase, also referred to herein as “the CYP27A1 enzyme,” introduces a hydroxyl group to the carbon at the 27 position in cholesterol, thereby producing 27OHC (See, FIG. 1). Human CYP27A1 gene, mRNA, and protein sequences are described in the art, and include, e.g., NCBI GenBank Accession Nos. NP_000775.1, NM_000784.3, XM_017003488.2, XP_016858977.1, and CCDS2423.1, UniProtKB Database Accession No. 002318, and NCBI Gene ID:1593. Inhibitors of the CYP27A1 enzyme include, but are not limited to, anastrozole, bicalutamide, posaconazole, fadrozole, dexmedetomidine, and ravuconazole (see, Mast et al., Mol. Pharmacal., 2015 88(3):428-36). In some embodiments, a 27OHC inhibitor inhibits the activity of 27OHC by sequestering and/or eliminating 27OHC before 27OHC acts on a target molecule or accumulates in the central nervous system.

The terms “nucleic acid” and “polynucleotide” are used interchangeably herein and refer to deoxyribonucleotides or ribonucleotides and polymers thereof in either single- or double-stranded form, and complements thereof. In some embodiments, the polynucleotide is DNA (e.g., genomic DNA or cDNA). In some embodiments, the polynucleotide is RNA (e.g., mRNA). Unless otherwise indicated, a particular nucleic acid sequence also implicitly encompasses conservatively modified variants thereof (e.g., degenerate codon substitutions), polymorphic variants (e.g., SNPs), splice variants, and nucleic acid sequences encoding truncated forms of proteins, complementary sequences, as well as the sequence explicitly indicated.

The terms “protein” and “polypeptide” are used interchangeably herein and refer to a polymer of amino acid residues. As used herein, the terms encompass amino acid chains of any length, including full-length proteins and truncated proteins.

As used herein, the term “compound” refers to any molecule, either naturally occurring or synthetic, e.g., peptide, protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, peptide, circular peptide, peptidomimetic, lipid, fatty acid, siRNA, micro RNA (miRNA), polynucleotide, oligonucleotide, etc.

As used herein, a “derivative” refers to a compound that is a structural derivative of a parent compound, in which one or more atoms or functional groups is different from the parent compound. In some embodiments, a derivative has comparable or superior stability, solubility, efficacy, half-life, and the like as compared to the parent compound.

As used herein, a “sample” refers to a bodily tissue or fluid obtained from a human or non-human mammalian subject. In some embodiments, a sample comprises blood, blood fractions, or blood products (e.g., serum, plasma, platelets, red blood cells, peripheral blood mononuclear cells, and the like), sputum or saliva, stool, urine, other biological fluids (e.g., lymph, saliva, prostatic fluid, gastric fluid, intestinal fluid, renal fluid, lung fluid, cerebrospinal fluid, and the like), tissue (e.g., kidney, spleen, lung, liver, heart, brain, nervous tissue, thyroid, eye, skeletal muscle, cartilage, or bone tissue), or cultured cells (e.g., primary cultures, explants, transformed cells, or stem cells). In some embodiments, a sample comprises blood. In some embodiments, a sample comprises cerebrospinal fluid (CSF).

A “subject” is a mammal, in some embodiments, a human. Mammals can also include, but are not limited to, farm animals (e.g., cows, pigs, horses, chickens, etc.), sport animals, pets, primates, horses, dogs, cats, mice and rats. In some instances, the term “subject” is used interchangeably with the term “patient”, particularly when describing a subject to be treated or receive treatment.

The terms “delay” or “delaying,” as used herein in the context of progression of Alzheimer's disease or progression from MCI to Alzheimer's disease, refers to a cessation or slowing in the decline of one or more parameters of Alzheimer's disease (e.g., a decline in cognitive function) in a subject or a maintenance of a level of one or more parameters of Alzheimer's disease (e.g., maintenance of a level of cognitive function) in the subject, e.g., as compared to a baseline such as an earlier assessment of the same subject. In some embodiments, a delay in the progression of Alzheimer's disease or a delay in the progression from MCI to Alzheimer's disease is determined by measuring the levels of one or more biomarkers in a sample from the subject. In some embodiments, a delay in the progression of Alzheimer's disease or a delay in the progression from MCI to Alzheimer's disease is measured by evaluating brain structure, e.g., MRI, CT or PET imaging. In some embodiments, a delay in the progression of Alzheimer's disease or a delay in the progression from MCI to Alzheimer's disease is determined by measuring cognitive function in the subject, e.g., using a standard scale or other methodology known in the art. Exemplary methods for determining or ranking cognitive function in a subject are described in Section III below.

The terms “reverse” or “reversing,” as used herein in the context of progression of Alzheimer's disease, refers to a reversal of decline or improvement in one or more parameters of Alzheimer's disease (e.g., a reversal in the decline of cognitive function) in a subject, e.g., as compared to a baseline such as an earlier assessment of the same subject. In some embodiments, a reversal in the progression of Alzheimer's disease is determined by measuring the levels of one or more biomarkers in the subject. In some embodiments, a reversal in the progression of Alzheimer's disease is measured by MRI, e.g., structural MRI. In some embodiments, a reversal in the progression of Alzheimer's disease is determined by measuring cognitive function in the subject, e.g., using a standard scale or other methodology known in the art. Exemplary methods for determining or ranking cognitive function in a subject are described herein under Section III.

As used herein, the terms “treatment,” “treating,” and “treat” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline (e.g., cognitive impairment); making the final point of degeneration less debilitating; and/or improving a subject's physical or mental well-being.

The term “pharmaceutical composition” refers to a composition suitable for administration to a subject. In general, a pharmaceutical composition is sterile, and preferably free of contaminants that are capable of eliciting an undesirable response with the subject. Pharmaceutical compositions can be designed for administration to subjects in need thereof via a number of different routes of administration, including oral, intravenous, intra-arterial, buccal, rectal, parenteral, intraperitoneal, intradermal, intratracheal, intramuscular, subcutaneous, inhalational, and the like.

### III. THERAPEUTIC METHODS

In one aspect, methods of treating a subject who has been diagnosed as having Alzheimer's disease (e.g., early-onset Alzheimer's disease or mild Alzheimer's disease) are provided. In some embodiments, the method comprises administering to a subject having Alzheimer's disease an oxysterol inhibitor such as a 27-hydroxycholesterol (27OHC) inhibitor. In some embodiments, the methods described herein relate to treating mild and/or moderate Alzheimer's disease. In some embodiments, the methods comprise treating a subject by delaying or reversing the progression of Alzheimer's disease in a subject diagnosed with Alzheimer's disease. In some embodiments, the methods comprise treating a subject by delaying the progression of Alzheimer's disease to severe Alzheimer's disease.

In another aspect, methods for treating a subject having Mild Cognitive Impairment (MCI) are provided. In some embodiments, the method comprises administering to a subject having MCI an oxysterol inhibitor such as a 27-hydroxycholesterol (27OHC) inhibitor. In some embodiments, the methods comprise treating a subject having MCI by delaying or preventing the onset of Alzheimer's disease in the subject.

### Oxysterol Inhibitors

In some embodiments, the therapeutic methods disclosed herein comprise administering an oxysterol inhibitor. In some embodiments, the oxysterol inhibitor is a 27OHC inhibitor. In some embodiments, a 27OHC inhibitor comprises a compound that inhibits or reduces the production of 27-hydroxycholesterol (27OHC) from cholesterol (e.g., in vitro or in vivo). In some embodiments, a 27OHC inhibitor comprises a compound that inhibits or reduces the production of 27OHC from cholesterol by the CYP27A1 enzyme.

In some embodiments, an oxysterol inhibitor inhibits or reduces the production of an oxysterol from cholesterol (e.g., in an in vitro or in vivo assay) by at least 5%, at least 10%, at least 15%, at least 20%, at least 25%, at least 30%, at least 40%, at least 50%, at least 60%, at least 70%, at least 80%, at least 90%, or more as compared to the level of production of the oxysterol from cholesterol in the absence of the oxysterol inhibitor. In some embodiments, the oxysterol inhibitor inhibits or reduces the activity of an enzyme that catalyses the formation of an oxysterol from cholesterol. As a non-limiting example, in some embodiments the oxysterol inhibitor is a 27OHC inhibitor that inhibits or reduces the activity of a CYP27A1 enzyme to catalyze the formation of 27-hydroxycholesterol (27OHC) from cholesterol (e.g., in vivo or in vitro). In some embodiments, a 27OHC inhibitor inhibits or reduces the activity of a CYP27A1 enzyme (e.g., in an in vitro or in vivo assay) by at least 5%, at least 10%, at least 15%, at least 20%, at least 25%, at least 30%, at least 40%, at least 50%, at least 60%, at least 70%, at least 80%, at least 90%, or more as compared to the level of activity of the CYP27A1 enzyme in the absence of the 27OHC inhibitor.

In some embodiments, a 27OHC inhibitor comprises a compound that inhibits the activity of 27OHC by sequestering and/or eliminating 27OHC before 27OHC acts on a target molecule or accumulates in the central nervous system. In some embodiments, a 27OHC inhibitor comprises a compound that sequesters 27OHC. In some embodiments, the compound is an antibody (e.g., an anti-27OHC antibody). The therapeutic use of antibodies to sequester proteins involved in cholesterol binding pathways is well known (e.g., ApoA1, Caveolin, CYP11A1, ORP1, etc). For example, in vivo administration in mice of an antibody (1D05) that binds PCSK9 prevented PCSK9 from binding to the low-density lipoprotein receptor (LDLr). The anti-PCSK9 antibody was observed to reduce circulating plasma LDL cholesterol levels in the treated mice (see, Ni et al., J. Lipid Research., (2011) 52:78-86 and Liang et al., J. Pharmacol Exp. Ther., (2012) 340:228-236). Additionally, human monoclonal antibodies such as evolocumab, alirocumab and bococizumab are known to bind PCSK9 and inhibit PCSK9 from binding to the LDLr for the treatment of LDL cholesterol levels. Accordingly, in some embodiments an anti-27OHC antibody can be used to sequester and/or eliminate 27OHC, e.g., to sequester 27OHC from circulating blood before 27OHC traverses the blood-brain barrier and accumulates in the central nervous system. Human antibodies that bind 27OHC are commercially available. For example, human 27-Hydroxyholestrol (27OHC) is commercially available as part of an ELISA Kit from MyBioSource, Catalog No. MBS109317.

In some embodiments, an oxysterol inhibitor inhibits the activity of 27OHC by sequestering 27OHC (e.g., in an in vitro or in vivo assay) by at least 5%, at least 10%, at least 15%, at least 20%, at least 25%, at least 30%, at least 40%, at least 50%, at least 60%, at least 70%, at least 80%, at least 90%, or more as compared to the level of 27OHC in the absence of the oxysterol inhibitor. As a non-limiting example, in some embodiments the oxysterol inhibitor is an anti-27OHC antibody. In some embodiments, a 27OHC inhibitor inhibits the activity of 27OHC by eliminating 27OHC (e.g., from blood, plasma, or the central nervous system) by at least 5%, at least 10%, at least 15%, at least 20%, at least 25%, at least 30%, at least 40%, at least 50%, at least 60%, at least 70%, at least 80%, at least 90%, or more as compared to the level of 27OHC in the absence of the oxysterol inhibitor.

In some embodiments, the oxysterol inhibitor comprises an aromatase inhibitor. As used herein, an “aromatase inhibitor” refers to a compound that inhibits the conversion of androgens into estrogen or blocks the activity of aromatase. In some embodiments, the aromatase inhibitor is anastrozole, exemestane, letrozole, vorozole, formestane, testolactone, aminoglutethimide, 1,4,6-androstatrien-3,17-dione, 4-androstene-3,6,17-trione, fadrozole, dexmedetomidine, ravuconazole, or a derivative thereof.

In some embodiments, the oxysterol inhibitor (e.g., the 27OHC inhibitor) comprises a compound selected from the group consisting of anastrozole, exemestane, letrozole, vorozole, formestane, testolactone, aminoglutethimide, 1,4,6-androstatrien-3,17-dione, 4-androstene-3,6,17-triune, bicalutamide, posaconazole, fadrozole, dexmedetomidine, ravuconazole, and derivatives or combinations thereof. In some embodiments, the oxysterol inhibitor (e.g., the 27OHC inhibitor) comprises anastrozole.

In some embodiments, determining whether a compound is an oxysterol inhibitor can be accomplished by measuring the production of an oxysterol from cholesterol in the presence of a suitable enzyme (e.g., a cytochrome P450 oxidase) and in the absence or presence of the compound and detecting a decrease in the production of oxysterol in the presence of the compound. For example, in some embodiments, determining whether a compound is a 27OHC inhibitor can be performed by measuring the production of 27OHC molecules from cholesterol by incubating the ‘test’ compound and purified CYP27A1 enzyme under test conditions. For example, recombinant, purified CYP27A1 may be prepared in vitro and incubated with the test compound to determine whether the test compound affects production of 27OHC molecules (see, Mast et al., Mol. Pharmacology, 2015, 88:428-436). In some embodiments, the test compound can be administered to a subject in vivo and plasma and/or blood levels of 27OHC can be measured after administration of the test compound to the subject (supra). In some embodiments, a compound that blocks or reduces the activity of CYP27A1 by at least 5%, at least 10%, at least 15%, at least 20%, at least 25%, at least 30%, at least 40%, at least 50%, at least 60%, at least 70%, at least 80%, at least 90%, or more, is determined to be a 27OHC inhibitor. In some embodiments, the 27OHC inhibitor blocks or reduces the activity of CYP27A1 by at least about 5%, at least about 10%, at least about 15%, at least about 20%, at least 25%, at least 30%, at least 40%, at least 50%, at least 60%, at least 70%, at least 80%, at least 90%, or more, in viva. In some embodiments, the 27OHC inhibitor blocks or reduces the activity of CYP27A1 in an amount from about 5% to about 95% in vivo.

In some embodiments, an oxysterol inhibitor such as a 27OHC inhibitor is administered in a therapeutically effective amount. As used herein, the term “therapeutically effective amount” refers to that amount of an agent (e.g., a 27OHC inhibitor or pharmaceutical composition comprising a 27OHC inhibitor) being administered that will treat to some extent a disease, disorder, or condition, e.g., relieve one or more of the symptoms of the disease, (i.e., cognitive impairment), being treated, and/or an amount that will prevent, to some extent, one or more of the symptoms of the disease (e.g., cognitive impairment) that the subject being treated has or is at risk of developing. In some embodiments, a daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated (e.g., mild, moderate or severe Alzheimer's disease), and the agent being employed. The dose will also be determined by the existence, nature, and extent of any adverse side-effects that accompany the administration of a particular agent in a particular patient. Determination of the proper dosage for a particular situation is within the skill of the practitioner. In some embodiments, treatment is initiated with smaller dosages which are less than the optimum dose of the compound. Thereafter, the dosage is increased by small increments until the optimum effect under the circumstances is reached. For convenience, the total daily dosage may be divided and administered in portions during the day, if desired. In some embodiments, the oxysterol inhibitor (e.g., 27OHC inhibitor) is administered to a subject for a period of at least 1 month, 2 months, 3 months, 4 months, 5 months, 6 months, 7 months, 8 months, 9 months, 10 months, 11 months, 12 months, or longer. In some embodiments, the oxysterol inhibitor (e.g., 27OHC inhibitor) is administered for an indefinite period of time. In some embodiments, the oxysterol inhibitor (e.g., 27OHC inhibitor) is administered for the rest of the subject's life or until administration of the inhibitor no longer provides a therapeutic benefit.

In the practice of the therapeutic methods described herein, an oxysterol inhibitor (e.g., a 27OHC inhibitor) can be administered according to any suitable method. Methods of administration include, for example, intravenous, intra-arterial, intrathecal, intraspinal, intraperitoneal, intramuscular, intranasal, subcutaneous, oral, topical, or inhalational administration.

In some embodiments, treatment with an oxysterol inhibitor as disclosed herein is combined with one or more other therapies. For example, in some embodiments, treatment with an oxysterol inhibitor as disclosed herein is combined with lifestyle changes, such as a low cholesterol diet, or treatment with an acetylcholinesterase inhibitor (ChEI).

### Subject Populations

In some embodiments, the subject to be treated is a human. In some embodiments, the subject is an adult human at least 30 years of age. In some embodiments, the subject is an adult human at least 65 years of age. In some embodiments, the subject is a human who has been diagnosed with Mild Cognitive Impairment or who is suspected of having Mild Cognitive Impairment. In some embodiments, the subject is a human who has been diagnosed with mild or moderate Alzheimer's disease. In some embodiments, the subject is a human who has been diagnosed with early-onset Alzheimer's disease. In some embodiments, the subject is a human who has been diagnosed with severe Alzheimer's disease.

Delaying or reversing progression of Alzheimer's disease

In some embodiments, the methods disclosed herein comprise delaying or reversing the progression of Alzheimer's disease in a subject having Alzheimer's disease (e.g., delaying the progression of Alzheimer's disease in a subject having mild Alzheimer's disease, moderate Alzheimer's disease, or severe Alzheimer's disease, or reversing the progression of Alzheimer's disease to a less severe form, e.g., from moderate to mild). In some embodiments, the progression of Alzheimer's disease is measured using one or more standard tests or scales for measuring cognitive function and/or other parameters of Alzheimer's disease, e.g., as described below. In some embodiments, the progression of Alzheimer's disease is measured by detecting the level of one or more biomarkers in the subject, e.g., as described below. In some embodiments, the progression of Alzheimer's disease is measured by magnetic resonance imaging (MRI), e.g., as described below.
Delayed progression of Alzheimer's disease

In some embodiments, administration of an oxysterol inhibitor (e.g., a 27OHC inhibitor) delays progression of Alzheimer's disease in a subject having Alzheimer's disease if there is a slowing of cognitive decline in the subject. In some embodiments, cognitive decline is determined using a standard scale (e.g., as described below) or measuring one or more activities of daily living (ADLs) over a period of time (e.g., between 1 and 6 months or between 1 month and 1 year) after administration of an oxysterol inhibitor (e.g., 27OHC inhibitor) to the subject. As a non-limiting example, the Alzheimer's Disease Functional Assessment of Change Scale (ADFACS) is a 16-item functional assessment based on both basic ADLs and instrumental ADLs (IADLs), in which each of the basic ADL items is scored on a scale of 0 (no impairment) to 4 (severe impairment) and each IADL item is scored on a scale ranging from 0 (no impairment) to 3 (severe impairment). See, Boustani et al., Screening for Dementia, Systematic Evidence Reviews, No. 20, 2003. In some embodiments, delayed progression of Alzheimer's disease includes maintaining a standard scale score or ranking of one or more ADLs measured over the period of time after administration of a 27OHC inhibitor to the subject. Thus, in some instances, progression of Alzheimer's disease is delayed if there is not a decline in one or more ADLs or standard scale score over the period of time during which the one or more ADLs are measured after administration of the 27OHC inhibitor.

In some instances, cognitive decline is measured by comparing a standard scale score or ranking of cognitive function (e.g., based on one or more ADLs) in the subject prior to administration of the oxysterol inhibitor (e.g., 27OHC inhibitor) as compared to the standard scale score or ranking of cognitive function after the onset of administration of the inhibitor to the subject (e.g., measured between 1 and 6 months after the inhibitor is first administered). In some embodiments, progression of Alzheimer's disease is delayed if the standard scale score or ranking of cognitive function after administration of the oxysterol inhibitor (e.g., 27OHC inhibitor) to the subject is the same as or not worse than the standard scale score or ranking of cognitive function prior to administration of the inhibitor to the subject.

In some instances, cognitive decline is measured by comparing a standard scale score or ranking of cognitive function (e.g., based on one or more ADLs) in a subject after administration of the oxysterol inhibitor (e.g., 27OHC inhibitor) to the subject (e.g., between 1 and 6 months after administration) as compared to cognitive function measured in a similarly situated subject (e.g., same gender, age, and ethnicity) who did not receive a therapeutically effective amount of the inhibitor.

In some instances, cognitive decline is measured by assessing the brain structure of a subject, e.g., using magnetic resonance imaging (MRI), computed tomography (CT), or positron emission tomography (PET) imaging. In some embodiments, cognitive decline is measured by comparing the brain structure of a subject having Alzheimer's disease (e.g., a MRI scan of the brain of the subject) prior to administration of the oxysterol inhibitor (e.g.,27OHC inhibitor) to the brain structure of the subject after administration of the oxysterol inhibitor to the subject (e.g., measured between 1 and 6 months after the oxysterol inhibitor is first administered). In some embodiments, progression of Alzheimer's disease is delayed if the brain structure of the subject after administration of the oxysterol inhibitor (e.g., 27OHC inhibitor) is the same as, or not worse, than the brain structure prior to administration of the oxysterol inhibitor to the subject.

In some instances, cognitive decline is measured by comparing the brain structure of a subject (e.g., a MRI scan of the brain of a subject) after administration of the oxysterol inhibitor (e.g., 27OHC inhibitor) to the subject (e.g., between 1 and 6 months after first administration) to the brain structure of a control subject (e.g., a MRI brain scan of a similarly situated subject (e.g., same gender, age, and ethnicity) who has not been administered the oxysterol inhibitor).

In some instances, cognitive decline is measured by assessing the level of one or more biomarkers in a subject (e.g., a biomarker as disclosed below). For example, in some embodiments, cognitive decline is assessed by measuring the level of one or more biomarkers in a sample from a subject prior to administration of the oxysterol inhibitor (e.g., 27OHC inhibitor) to establish a baseline level and then measuring the level of the one or more biomarkers in a sample from the subject after administration of the oxysteral inhibitor to the subject (e.g., measured between 1 and 6 months after the oxysterol inhibitor is first administered).

In some instances, cognitive decline is measured by comparing the level of one or more biomarkers in a sample from a subject after administration of the oxysterol inhibitor (e.g., 27OHC inhibitor) to the subject (e.g., between 1 and 6 months after first administration) to the level of the one or more biomarkers in a sample from a similarly situated subject (e.g., same gender, age, and ethnicity) who has not been administered the oxysterol inhibitor.

In some aspects, delaying progression of Alzheimer's disease in a subject having Alzheimer's disease can include prolonging the period of time that the subject has mild or moderate Alzheimer's disease (e.g., by extending the period of mild or moderate Alzheimer's disease by 6 months, a year, or more) as compared to average pendency's known in the art that typically reflect the length of “mild” or “moderate” Alzheimer's disease. In some embodiments, the methods described herein delay the progression of Alzheimer's disease in a subject having mild or moderate Alzheimer's disease to severe Alzheimer's disease. In some embodiments, the methods described herein delay the progression of Alzheimer's disease in a subject having mild Alzheimer's disease to moderate Alzheimer's disease.
Reversing progression of Alzheimer's disease

In some embodiments, administration of an oxysterol inhibitor (e.g., a 27OHC inhibitor) reverses progression of Alzheimer's disease in a subject having Alzheimer's disease if there is an improvement in cognitive function in the subject. In some embodiments, improvement in cognitive function is determined using a standard scale (e.g., the Alzheimer's Disease Functional Assessment of Change Scale (ADFACS)) or measuring one or more ADLs over a period of time (e.g., between 1 and 6 months or between 1 month and 1 year), after administration of an oxysterol inhibitor (e.g., 27OHC inhibitor) to the subject. In some embodiments, reversing progression of Alzheimer's disease includes improving a standard scale score or ranking of one or more ADLs measured over a period of time after administration of the inhibitor to the subject. Thus, in some instances, the progression of Alzheimer's disease in a subject is reversed if the one or more ADLs or standard scale score improves by one or more points (e.g., by 1, 2, 3, 4, 5, 6, 7, 8, 9, 10 points or more) during the period of time in which the one or more ADLs are measured after administration of the inhibitor to the subject.

In some instances, improvement in cognitive function is measured by comparing a standard scale score or ranking of cognitive function (e.g., based on one or more ADLs) in the subject prior to administration of the oxysterol inhibitor (e.g., 27OHC inhibitor) as compared to the standard scale score or ranking of cognitive function after the onset of administration of the 27OHC inhibitor to the subject (e.g., measured between 1 and 6 months after the inhibitor is first administered). In some embodiments, progression of Alzheimer's disease is reversed when the standard scale score or ranking of one or more ADLs has improved by at least one point during the period in which the standard scale score or ADL is measured after administration of the inhibitor to the subject. In some embodiments, the standard scale score can improve by 1, 2, 3, 4, 5, 6, 7, 8, 9, 10, or more points. In some embodiments, an individual ADL can improve by one or more points. In some embodiments, two or more ADLs in a standard score are each improved by at least one point. In some embodiments, the improvement in cognitive function is maintained for at least 1, 2, 3, 4, 5, or 6 months after administration of the inhibitor to the subject. In some embodiments, the improvement in cognitive function is maintained for as long as the subject is treated with the 27OHC inhibitor.

In some instances, improvement in cognitive function is measured by comparing brain structure (e.g., using MRI, CT, or PET imaging) of a subject having Alzheimer's disease prior to administration of the oxysterol inhibitor (e.g., 27OHC inhibitor) to the brain structure of the subject after administration of the oxysterol inhibitor to the subject (e.g., measured between 1 and 6 months after the oxysterol inhibitor is first administered). In some embodiments, improvement in cognitive function is achieved if the brain structure of the subject after administration of the oxysterol inhibitor (e.g., 27OHC inhibitor) is the same as, or improved, as compared to the brain structure of the subject prior to administration of the oxysterol inhibitor to the subject.

In some instances, improvement in cognitive function is measured by comparing the brain structure of a subject (e.g., using MRI, PET, or CT imaging) after administration of the oxysterol inhibitor (e.g., 27OHC inhibitor) to the subject (e.g., between 1 and 6 months after first administration) to the brain structure of a similarly situated subject (e.g., same gender, age, and ethnicity) who has not been administered the oxysterol inhibitor.

In some instances, improvement in cognitive function is assessed by measuring the level of one or more biomarkers in a sample from a subject prior to administration of the oxysterol inhibitor (e.g., 27OHC inhibitor), measuring the level of the one or more biomarkers in a sample from the subject after administration of the oxysterol inhibitor to the subject (e.g., measured between 1 and 6 months after the oxysterol inhibitor is first administered), and comparing the the level of the one or more biomarkers in the subject after administration of the oxysterol inhibitor to the level of the one or more biomarkers in the subject prior to administration of the oxysterol inhibitor to the subject.

In some instances, improvement in cognitive function is assessed by measuring the level of one or more biomarkers in a sample from a subject after administration of the oxysterol inhibitor (e.g., 27OHC inhibitor) to the subject (e.g., between 1 and 6 months after first administration) and comparing the subject's level to the level of the one or more biomarkers in a sample from a similarly situated subject (e.g., same gender, age, and ethnicity) who was not administered the oxysterol inhibitor.

In some aspects, reversing progression of Alzheimer's disease in a subject having Alzheimer's disease can include prolonging the period of time that the subject has mild or moderate Alzheimer's disease (e.g., by extending the period of mild or moderate Alzheimer's disease by 6 months, a year, or more) as compared to average pendency's known in the art that typically reflect the length of “mild” or “moderate” Alzheimer's disease. In some embodiments, the methods described herein reverse the progression of moderate Alzheimer's disease in a subject to mild Alzheimer's disease. In some embodiments, the methods described herein reverse the progression of severe Alzheimer's disease in a subject to moderate Alzheimer's disease.

Delaying onset of Alzheimer's disease in a subject having mild cognitive impairment (MCI)

In some embodiments, the methods disclosed herein comprise delaying the onset of Alzheimer's disease in a subject having Mild Cognitive Impairment (MCI). In some embodiments, the delay in onset of Alzheimer's disease is measured using one or more standard tests or scales for measuring cognitive function and/or other parameters of Alzheimer's disease, e.g., as described below. In some embodiments, the delay in onset of Alzheimer's disease is measured by detecting the level of one or more biomarkers in the subject, e.g., as described below. In some embodiments, the delay in onset of Alzheimer's disease is measured by magnetic resonance imaging (MRI), e.g., as described below.

In some embodiments, administration of an oxysterol inhibitor (e.g., a 27OHC inhibitor) delays the onset of Alzheimer's disease in a subject having MCI if there is a slowing of cognitive decline, a maintenance of cognitive function, or an improvement in cognitive function in the subject. In some embodiments, cognitive function is determined using a standard scale (e.g., the Alzheimer's Disease Functional Assessment of Change Scale (ADFACS)) or measuring one or more ADLs over a period of time (e.g., between 1 and 6 months or between 1 month and 1 year), after the onset of administering an oxysterol inhibitor (e.g., 27OHC inhibitor) to the subject having MCI. In some embodiments, the onset of Alzheimer's disease is delayed if the subject maintains a standard scale score or ranking of one or more ADLs measured over the period of time after onset of administration of a an oxysterol inhibitor. In some embodiments, the onset of Alzheimer's disease is delayed if the subject does not exhibit a decrease or decline in the one or more ADLs or standard scale score over the period of time during which the one or more ADLs are measured after the onset of administration of an oxysterol inhibitor. In some embodiments, the onset of Alzheimer's disease is delayed if the subject exhibits an increase or improvement in the one or more ADLs or standard scale score over the period of time during which the one or more ADLs are measured after the onset of administration of an oxysteral inhibitor.

In some instances, cognitive function is measured by comparing a standard scale score or ranking of cognitive function (e.g., based on one or more ADLs) in the subject prior to administration of the inhibitor as compared to the standard scale score or ranking of cognitive function after the onset of administration of the inhibitor to the subject having MCI (e.g., measured between 1 and 6 months after administration of the inhibitor). In some embodiments, the onset of Alzheimer's disease is delayed if the standard scale score or ranking of cognitive function after administration of the oxysterol inhibitor (e.g., 27OHC inhibitor) to the subject having MCI is better than, the same as, or not worse than the standard scale score or ranking of cognitive function prior to administration of the inhibitor to the subject having MCI.

In some instances, cognitive function is measured by comparing a standard scale score or ranking of cognitive function (e.g., based on one or more ADLs) in a subject having MCI after administration of the inhibitor to the subject (e.g., between 1 and 6 months after administration) as compared to cognitive function measured in a similarly situated subject (e.g., same gender, age, ethnicity and having MCI) who did not receive a therapeutically effective amount of the inhibitor.

In some embodiments, delay in onset of Alzheimer's disease in a subject having MCI is determined by assessing the brain structure of the subject, e.g., using MRI, CT, or PET imaging as disclosed herein. In some embodiments, delay in onset of Alzheimer's disease is measured by comparing the brain structure (e.g., using MRI, CT, or PET imaging) of a subject having MCI prior to administration of the oxysterol inhibitor (e.g., 27OHC inhibitor) to the brain structure of the subject after administration of the oxysterol inhibitor to the subject (e.g., measured between 1 and 6 months after the oxysterol inhibitor is first administered). In some embodiments, there is a delay in onset of Alzheimer's disease if the brain structure of the subject does not worsen after administration of the oxysterol inhibitor (e.g., 27OHC inhibitor) as compared to the brain structure of the subject prior to administration of the oxysterol inhibitor to the subject. For example, in some embodiments, there is a delay in onset of Alzheimer's disease if there is not a decrease in hippocampal volume in the subject after administration of the oxysterol inhibitor as compared to prior to administration of the oxysterol inhibitor to the subject.

In some aspects, delaying the onset of Alzheimer's disease in a subject having MCI comprises prolonging the period of time that the subject having MCI has no symptoms of Alzheimer's disease or symptoms corresponding to mild Alzheimer's disease (e.g., by extending the period of MCI by 6 months, a year, or more), for example as compared to an average length of time from diagnosis of MCI to diagnosis to Alzheimer's disease for similarly situated subjects. In some embodiments, delaying the onset of Alzheimer's disease in a subject having MCI comprises prolonging the period of time that the subject is not clinically diagnosed as having Alzheimer's disease. In some embodiments, the methods described herein delay the onset of Alzheimer's disease in a subject having MCI by at least 3, 4, 5, 6, or more months, or longer. In some embodiments, the methods described herein delay the onset of Alzheimer's disease in a subject having MCI for at least 1 year or longer.

Preventing and/or delaying onset of Alzheimer's disease and/or MCI in a subject who is at least 60 years old and/or has a cholesterol level of at least 200 mg/dL

In some embodiments, the methods disclosed herein comprise preventing and/or delaying the onset of Alzheimer's disease and/or Mild Cognitive Impairment in a subject who is at least 60 years old (e.g., at least 65, 70, 75, 80, 85, 90, 95, or 100 years old; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old) and/or has a cholesterol level of 200 mg/dL or above (e.g., 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/dL) by administering to the subject an oxysterol inhibitor.

In certain embodiments, the methods disclosed herein comprise preventing and/or delaying the onset of Alzheimer's disease and/or Mild Cognitive Impairment in a subject who is at least 60 years old (e.g., at least 65, 70, 75, 80, 85, 90, 95, or 100 years old; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old) by administering to the subject an oxysterol inhibitor. In some embodiments, the subject who is at least 60 years old (e.g., at least 65, 70, 75, 80, 85, 90, 95, or 100 years old; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old) also has a cholesterol level of 200 mg/dL or above (e.g., 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/dL).

In certain embodiments, the methods disclosed herein comprise preventing and/or delaying the onset of Alzheimer's disease and/or Mild Cognitive Impairment in a subject who has a cholesterol level of 200 mg/dL or above (e.g., 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/dL) by administering to the subject an oxysterol inhibitor. in some embodiments, the subject, who has a cholesterol level of 200 mg/dL or above (e.g., 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mg/dL), is also at least 60 years old (e.g., at least 65, 70, 75, 80, 85, 90, 95, or 100 years old; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old).

In some embodiments of this aspect, the oxysterol inhibitor is a 27OHC inhibitor. In some embodiments, the 27OHC inhibitor inhibits the production of 27-hydroxycholesterol by the enzyme CYP27A1.

In some embodiments of this aspect, the oxysterol inhibitor is an aromatase inhibitor.

In some embodiments of this aspect, the oxysterol inhibitor is selected from the group consisting of anastrozole, exemestane, letrozole, vorozole, formestane, testolactone, a minoglutethimide, 1,4,6-androstatrien-3,17-dione, 4-androstene-3,6,17-trione, bicalutamide, posaconazole, fadrozole, dexmedetomidine, ravuconazole, and derivatives or combinations thereof. In particular embodiments, the oxysterol inhibitor is anastrozole.

In some embodiments of this aspect, administration of the oxysterol inhibitor to the subject results in an increased level of expression of rhotekin 2 (RTKN2) in the subject.

Standard Scales for Alzheimer's disease

In some embodiments, the efficacy of treatment is measured by assessing cognitive function or cognitive ability in the subject being treated. In some embodiments, cognitive function or cognitive ability is assessed using a “standard scale” known in the art by which to measure the extent and/or progression of Alzheimer's disease in a subject. Standard scales are described, for example, in Boustani et al., Screening for Dementia, Systematic. Evidence Reviews, No. 20, 2003. Examples of standard scales for assessing Alzheimer's disease include, but are not limited to, mini-mental state examination (MMSE) (J Psychiatr Res. 1975;12(3):189-198); Alzheimer's Disease Assessment Scale-Cognitive subscale (ADAS-Cog) (Brain Imaging Behav. 2012 Dec; 6(4): 10.1007/s11682-012-9166-3); Clinician's Global Impression of Change (CGIC) (Psychiatry (Edgmont). 2007 Jul; 4(7): 28-37); Clinician's Interview-Based Impression (CIBI) (Knopman et al., Neurology, 1994, 44(12), doi.org/10.1212/WNL.44.12.2315); Clinician's Interview-Based Impression of Change and Clinician's Interview-Based Impression of Change with caregiver input (CIBIC-Plus); Alzheimer's Disease Functional Assessment of Change Scale (ADFACS); Clinical Dementia Rating Scale (CDR); Gottfries-Brane-Steen Scale (GBS); Interview for Deterioration in Daily living in Dementia Scale (IDDD); Neuropsychiatry Inventory Scale (NPI); Progressive Deterioration Scale (PDS); and Resource Utilization in Dementia Questionnaire Scale (RUD).

It is contemplated herein that any one or more of the standard scales disclosed herein may be used to determine progression of Alzheimer's disease in an Alzheimer's subject or in a subject having MCI for the onset of Alzheimer's disease. In one embodiment, one or more parameters or characteristics within a standard scale or the overall score obtained using a standard scale may be used to determine whether the subject's Alzheimer's disease progression is delayed, reversed, halted, or improved after administration of an oxysterol inhibitor (e.g., 27OHC inhibitor) to the subject. In another embodiment, one or more parameters or characteristics within a standard scale or the overall score obtained using the standard scale may be used to determine whether a subject having MCI has developed Alzheimer's disease after administration of an oxysterol inhibitor (e.g., 27OHC inhibitor) to the subject. In yet another embodiment, one or more parameters or characteristics within a standard scale or the overall score obtained using the standard scale may be used to determine whether a subject having MCI, and who is treated with an inhibitor as disclosed herein, has improved parameters, characteristics, or an overall score as compared to an overall score obtained using the same standard scale prior to administration of the inhibitor to the subject.

In some embodiments, a standard scale as disclosed herein is used to determine whether a subject's Alzheimer's disease progression is reversed, halted, or improved after administration of an oxysterol inhibitor (e.g., 27OHC inhibitor) to the subject. In some embodiments, a standard scale or the overall score obtained using the standard scale is used to determine whether a subject having MCI has developed Alzheimer's disease after administration of an oxysterol inhibitor (e.g., 27OHC inhibitor) to the subject. In some embodiments, a standard scale or the overall score obtained using the standard scale is used to determine whether a subject having MCI, and who is treated with an inhibitor as disclosed herein, has an improved overall standard score as compared to an overall standard score obtained prior to administration of the inhibitor to the subject.

In some embodiments, the extent and/or progression of Alzheimer's disease in a subject is determined by the Alzheimer's Disease Functional Assessment of Change Scale (ADFACS). The ADFACS is a 16-item functional assessment test based on activities of daily living (ADLs) (e.g., basic self-care such as bathing, dressing and grooming, eating, toilet use, control of urine and bowels, moving about, etc.,) and instrumental activities of daily living (IADLs) (e.g., managing money, managing medication, cooking, using appliances, housekeeping, shopping, etc.). Typically, a physician obtains information directly from the subject or a caregiver to the subject. Each ADL is scored on a range of 0 (no impairment) to 4 (severe impairment) and each IADL is scored on a range of 0 (no impairment) to 3 (severe impairment). The total score for the ADFACS scale is from 0 to 54. Those with higher scores are considered less impaired.

In some embodiments, the extent and/or progression of Alzheimer's disease in a subject is determined by a clinical dementia rating (CDR) scale. The CDR measures six areas of activity including: memory; orientation; judgement and problem solving; community affairs; home and hobbies; and personal care. Each area of activity is scored on a range from 0 (no impairment) to 3 (severe impairment) (see, Hughes et al., Br. J. Psychiatry, 1982, 140:566-572).

In some embodiments, the extent and/or progression of Alzheimer's disease in a subject is determined by the Gottfries-Brane-Steen (GBS) scale. The GBS scale is a 27-item scale for rating Alzheimer's disease based on a semi-structured interview by a physician, with both the subject (e.g., patient) and caregiver to the subject. (see., Gottfries et al., Arch. Gerontol. Geriatr., 1982, 1:311-30 and Brane et al., Dement. Geriatr. Cogn. Disord., 2001, 12:1-14) The GBS assesses 4 areas: intellectual impairment (orientation, memory, concentration (12 items)), self-care motor function (6 items), emotional reaction (3 items), and behavioral symptoms (6 items). A 7-point scoring system from 0 to 6 is used for each of the 27 items, giving a total score range of 0 to 162 points, with an increase in score representing clinical deterioration.

In some embodiments, the extent and/or progression of Alzheimer's disease in a subject is determined by an interview for deterioration in daily living in dementia (IDDD) scale. The IDDD scale assesses functional disability in basic ADLs (16 items) and IADLs (17 items) of subjects (e.g., patients) living in the community. A caregiver assesses the patients' severity of impairment in each item using a 7-point scale, where 1 to 2 points denotes no or slight impairment, 3 to 4 points denotes mild impairment, 5 to 6 points denotes moderate impairment, and 7 points denotes severe impairment. Thus, the IDDD scale has a total score range of 33 to 231 points.

In some embodiments, the extent and/or progression of Alzheimer's disease in a subject is determined using the Neuropsychiatry inventory scale (NPI). The NPI scale evaluates the frequency and severity of 10 neuropsychiatric disturbances that occur frequently in dementia: agitation, irritability, anxiety, dysphoria, hallucinations, delusions, apathy, euphoria, disinhibition, and aberrant motor behavior. Each item on the NPI is scored on a 1 to 4 point frequency scale and a 1 to 3 point severity scale. The severity score is then multiplied by the frequency score, resulting in a total score ranging from 10 to 120 points (see, Cummings et al., Neurology, 1994, 44:2308-2314).

In some embodiments, the extent and/or progression of Alzheimer's disease in a subject is determined by the Progressive Deterioration Scale (PDS). The PDS scale is a self-administered scale for caregivers of a subject (e.g., patient) that examines the ability of patients to accomplish basic ADLs and IADLs in 11 areas (see, DeJong et al., Clin. Ther., 1989, 11:545-54). Each item is scored using a 100mm bipolar visual analogue scale, then a total score range from 0 to 100 is derived from the average across the items (Demers et al., J. Geriatr. Psychiatry Neural., 2000, 13:161-69.

In some embodiments, the extent and/or progression of Alzheimer's disease in a subject is determined by the Resource Utilization in Dementia Questionnaire Scale (RUD). The RUD scale is completed by caregivers of a subject (e.g., patient) that complies data on the use of social services, frequency and duration of hospitalizations, unscheduled contacts with health care professionals, use of concomitant medications by both the caregiver and the patient, amount of time the caregiver spends caring for the patient and missing work, and patients' use of medication (see, Burns et al., Br. J. Psychiatry, 1990, 157:72-76 and 92-94.

In some embodiments, the subject is assessed for cognitive function (e.g., changes in parameters or characteristics pertaining to Alzheimer's disease as measured according to a standard scale as disclosed herein) once or more, over a period of time after administration of the oxysterol inhibitor to the subject. In some embodiments, the subject is periodically assessed for cognitive function, e.g., over a period of days, weeks, or months after the onset of treatment with the oxysterol inhibitor. In some embodiments, the parameter, characteristic or standard scale is measured once a month, once a quarter, every six months and/or annually after the onset of administration of the inhibitor to the subject.

### Imaging Analysis

In some embodiments, the efficacy of treatment is measured by imaging the brain of the subject. For example, in some embodiments, magnetic resonance imaging (MRI), computed tomography (CT), or positron emission tomography (PET) is performed to evaluate brain structure. The use of imaging technologies for the clinical evaluation of subjects having Alzheimer's disease or MCI is described in the art. See, e.g., Johnson et al., Cold Spring Harb Perspect Med, 2012, 2(4):a00613; Jack et al., J. Magn. Reson Imaging, 2008, 27(4):685-691; and Vernuri and Jack, Alzheimer's Research and Therapy, 2010, 2:23 (doi.org/10.1186/alzrt47). For example, MRI can be used to quantify brain volume and to quantify global percentage change in brain volume (brain atrophy) at different timepoints. As another example, MRI can be used to measure reduction in hippocampal volume or increase in ventricular volume.

In some embodiments, delayed or reversed progression of Alzheimer's disease in a subject having Alzheimer's disease is determined by evaluating the brain structure of the subject using MRI, e.g., structural MRI (sMRI), after the subject is treated with an oxysterol inhibitor. In some embodiments, delayed onset of Alzheimer's disease in a subject having MCI is determined by evaluating the brain structure of the subject using MRI, e.g., sMRI, after the MCI subject is treated with an oxysterol inhibitor. In some embodiments, the results of the imaging (e.g., MRI) are compared to a baseline, e.g., from a control subject or from the subject prior to the onset of treatment.

### Biomarker Analysis

In some embodiments, the method comprises measuring in a sample from the subject one or more biomarkers for Alzheimer's disease. In some embodiments, the biomarker is a biomarker disclosed in U.S. Patent Application 62/432,091 or PCT Application PCT/US2017/065367 (incorporated herein by reference). As disclosed in PCT/US2017/065367, it has been found that the expression of rhotekin 2 (RTKN2) and other proteins, such as microtubule-associated Ser/Thr kinase 4 (MAST4), and the transcriptional activity forkhead box O1 (FOXO1) and amyloid precursor protein (APP)), are dysregulated in the brains of Alzheimer's disease subjects. In some embodiments, the biomarker is RKTN2.

In some embodiments, delayed or reversed progression of Alzheimer's disease in a subject haying Alzheimer's disease is determined by measuring the level of one or more biomarkers, such as RTKN2, MAST4, FOXO1, or APP, in a sample from a subject being treated with an oxysterol inhibitor (e.g., 27OHC inhibitor). In some embodiments, delayed onset of Alzheimer's disease in a subject having MCI is determined by measuring the level of one or more biomarkers, such as RTKN2, MAST4, FOXO1, or APP, in a sample from a subject being treated with an oxysterol inhibitor (e.g., 27OHC inhibitor). In some embodiments, the level of expression of RTKN2 mRNA or protein in the sample from the subject is increased by at least 10%, at least 20%, at least 30%, at least 40%, at least 50%, at least 60%, at least 70%, at least 80%, at least 90%, as compared to a reference value (e.g., the level of expression of RTKN2 mRNA or protein in a sample from the subject prior to the start of treatment).

In some embodiments, delayed or reversed progression of Alzheimer's disease in a subject having Alzheimer's disease is determined by measuring the transcriptional activity of one or more biomarkers, such as FOXO1 and/or APP, in a sample from a subject being treated with an oxysterol inhibitor (e.g., 27OHC inhibitor). In some embodiments, transcriptional activity of FOXO1 in the sample from the subject is modulated by at least 10%, at least 20%, at least 30%, at least 40%, at least 50%, at least 60%, at least 70%, at least 80%, at least 90%, as compared to a reference value (e.g., the transcriptional activity of FOX01 in a sample from the subject prior to the start of treatment).

In some embodiments, polynucleotide (e.g., mRNA) expression is measured using routine techniques such as reverse transcription polymerase chain reaction (RT-PCR), Real-Time reverse transcription polymerase chain reaction (Real-Time RT-PCR), semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, polynucleotide (e.g., mRNA) expression is measured by sequencing. Non-limiting examples of sequence analysis include Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and “next generation sequencing” methods, including but not limited to sequencing by synthesis (e.g., HiSeq™, MiSeq™, or Genome Analyzer, each available from IIlumina), sequencing by ligation (e.g., SOLiD™, Life Technologies), ion semiconductor sequencing (e.g., Ion Torrent™, Life Technologies), and pyrosequencing (e.g., 454′m sequencing, Roche Diagnostics). See, e.g., Liu et al., J. Biomed Biotechnol, 2012, 2012:251364, incorporated by reference herein. In some embodiments, polynucleotide expression is measuring using RNA-Seq technology. See, e.g., Finotello et al., Briefings in Functional Genomics, 2014, doi:10.1093/bfgp/e1u035; and Mortazavi et al., Nat Methods, 2008, 5:621-628.

A detectable moiety can be used in the assays described herein (direct or indirect detection). A wide variety of detectable moieties can be used, with the choice of label depending on the sensitivity required, ease of conjugation with the probe, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, metals, and the like.

Protein expression can be detected and quantified in a biological sample using routine techniques such as immunoassays, two-dimensional gel electrophoresis, and quantitative mass spectrometry that are known to those skilled in the art. Protein quantification techniques are generally described in “Strategies for Protein Quantitation,” Principles of Proteornics, 2nd Edition, R. Twyman, ed., Garland Science, 2013. In some embodiments, protein expression is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Blamed. Sci., 699:463-80 (1997)).

Specific immunological binding of the antibody to a protein can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein marker is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like.

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays can be automated or performed robotically, and the signal from multiple samples can be detected simultaneously. In some embodiments, the amount of signal can be quantified using an automated high-content imaging system. High-content imaging systems are commercially available (e.g., ImageXpress, Molecular Devices Inc., Sunnyvale, Calif.).

In some embodiments, protein expression is detected by quantitative mass spectrometry, for example but not limited to, spectral count MS, ion intensities MS, metabolic labeling (e.g., stable-isotope labeling with amino acids in cell culture (SILAC), enzymatic labeling, isotopic labeling (e.g., isotope-coded protein labeling (ICPL) or isotope-coded affinity tags (ICAT)), and isobaric labeling (e.g., tandem mass tag (TMT) or isobaric tags for absolute and relative quantification (iTRAQ)). See, e.g., Bantscheff et al., Anal Bioanal Chem, 2012, 404:949 (doi:10.1007/s00216-012-6203-4); and Nikolov et al., Methods in Molecular Biology, 2012, 893:85-100.

### Reference Values

In some embodiments, for assessing the efficacy of a treatment disclosed herein, a measurement from a subject as disclosed herein (e.g., using a standard scale, brain imaging, or biomarker analysis) is compared to a reference value. A variety of methods can be used to determine the reference value for a unit of measurement (e.g., standard scale, MRI, or biomarker) as described herein.

In one embodiment, a reference value for a unit of measurement (e.g., standard scale, MRI, or biomarker) as described herein is determined for the subject to be treated prior to the onset of treatment with an oxysterol inhibitor as disclosed herein. In some embodiments, the subject is assessed prior to the onset of treatment using a standard scale, MRI imaging, or biomarker analysis as disclosed herein to determine a “baseline value” for the subject, against which the subject is compared at one or more timepoints after the onset of treatment with an oxysterol inhibitor.

In another embodiment, a reference value for an MRI is determined by imaging the brain of a cognitively normal subject or population of subjects (e.g., subjects known not to have Alzheimer's disease or MCI) to determine characteristic brain features of a cognitively normal brain. In one embodiment, a reference value for a particular biomarker (e.g., RTKN2) is determined by assessing the level of that particular biomarker in samples from a cognitively normal subject or population of subjects (e.g., subjects known not to have Alzheimer's disease or MCI). As a non-limiting example, in one embodiment, a reference value is determined for a population of subjects (e.g., 10, 20, 50, 100, 200, 500 subjects or more) all known not to have Alzheimer's disease or MCI.

In another embodiment, a reference value for a unit of measurement (e.g., standard scale, MRI, or biomarker) as described herein is determined for a subject or population of subjects diagnosed as having the same form of Alzheimer's disease or MCI as the test subject. For example, in some embodiments, a reference value for an MRI is determined by imaging the brain of a subject or population of subjects diagnosed as having MCI or a particular form of Alzheimer's disease (e.g., mild or early-onset Alzheimer's disease) to determine characteristic brain features for the disease. In some embodiments, a reference value for a particular biomarker (e.g., RTKN2) is determined by assessing the level of that particular biomarker in samples from a subject or population of subjects diagnosed as having MCI or a particular form of Alzheimer's disease (e.g., mild or early-onset Alzheimer's disease). As a non-limiting example, in one embodiment, a reference value is determined for a population of subjects (e.g., 10, 20, 50, 100, 200, 500 subjects or more) all having MCI or a specific form of Alzheimer's disease.

In some embodiments, the population of subjects is matched to a test subject according to one or more patient characteristics such as age, sex, ethnicity, or other criteria. In some embodiments, the reference value is established using the same type of sample from the population of subjects (e.g., sample comprising blood or cerebrospinal fluid) as is used for assessing the level of the biomarker in the test subject. In some embodiments, the reference value for a unit of measurement (e.g., standard scale, MRI, or biomarker) as described herein is a measurement from the test subject, e.g., a measurement taken before the onset of treatment, or a prior measurement taken during the course of treatment.

Determination of particular threshold values for identifying a test subject as having Alzheimer's disease, selection of appropriate ranges, categories, stage of Alzheimer's disease, and the like are within the skill of those in the art guided by this disclosure. It will be understood that standard statistical methods may be employed by the practitioner in making such determinations. See, e.g., Principles of Biostatistics by Marcello Pagano et al. (Brook Cole; 20(X); and Fundamentals of Biostatistics by Bernard Rosner (Duxbury Press, 5th Ed, 1999).

### IV. PHARMACEUTICAL COMPOSITIONS AND KITS

In another aspect, pharmaceutical compositions and kits comprising an oxysterol inhibitor as disclosed herein are provided. In some embodiments, the pharmaceutical compositions and kits are for use in treating a subject having Alzheimer's disease (e.g., for delaying or reversing progression of Alzheimer's disease). In some embodiments, the pharmaceutical compositions and kits are for use in treating a subject having Mild Cognitive Impairment (MCI) (e.g., for delaying onset of Alzheimer's disease in a subject having MCI).

### Pharmaceutical Compositions

In some embodiments, pharmaceutical compositions comprising an oxysterol inhibitor are provided. In some embodiments, the oxysterol inhibitor is a 27OHC inhibitor. In some embodiments, the oxysterol inhibitor is an aromatase inhibitor that is anastrozole, exemestane, letrozole, vorozole, formestane, testolactone, aminoglutethimide, 1,4,6-androstatrien-3,17-dione, 4-androstene-3,6,17-triune, or a derivative thereof. In some embodiments, the oxysterol inhibitor comprises anastrozole, exemestane, letrozole, vorozole, formestane, testolactone, aminoglutethimide, 1,4,6-androstatrien-3,17-dione, 4-androstene-3,6,17-triune, bicalutamide, posaconazole, fadrozole, dexmedetomidine, ravuconazole, or derivatives or combinations thereof. In some embodiments, the oxysterol inhibitor is anastrozole.

Guidance for preparing formulations can be found in any number of handbooks for pharmaceutical preparation and formulation that are known to those of skill in the art. See, e.g., Remington: The Science and Practice of Pharmacy, 21st Edition, Philadelphia, Pa. Lippincott Williams & Wilkins, 2005.

In some embodiments, the pharmaceutical composition further comprises one or more pharmaceutically acceptable carriers, adjuvants, and/or vehicles appropriate for the particular route of administration for which the composition is to be employed. In some embodiments, the carrier, adjuvant, and/or vehicle is suitable for intravenous, intramuscular, oral, intraperitoneal, transdermal, topical, or subcutaneous administration. Pharmaceutically acceptable carriers are well-known in the art. See, e.g., Handbook of Pharmaceutical Excipients (5th ed., Ed. Rowe et of., Pharmaceutical Press, Washington, D.C.). Examples of pharmaceutically acceptable carriers include, but are not limited to, aqueous solutions, e.g., water or physiologically compatible buffers such as Fianks's solution, Ringer's solution, or physiological saline buffer.

Typically, a pharmaceutical composition for use in in vivo administration is sterile. Sterilization can be accomplished according to methods known in the art, e.g., heat sterilization, steam sterilization, sterile filtration, or irradiation.

The dosages of pharmaceutical compositions of the disclosure may vary depending on the particular use envisioned. The determination of the appropriate dosage or route of administration is well within the skill of one in the art. Suitable dosages are also described in Section III above.

### Kits

In some embodiments, kits comprising an oxysterol inhibitor or a pharmaceutical composition comprising an oxysterol inhibitor are provided. In some embodiments, the oxysterol inhibitor is a 27OHC inhibitor. In some embodiments, the oxysterol inhibitor is an aromatase inhibitor that is anastrozole, exemestane, letrozole, vorozole, formestane, testolactone, aminoglutethimide, 1,4,6-androstatrien-3,17-dione, 4-androstene-3,6,17-triune, or a derivative thereof. In some embodiments, the oxysterol inhibitor comprises anastrozole, exemestane, letrozole, vorozole, formestane, testolactone, aminoglutethimide, 1,4,6-androstatrien-3,17-dione, 4-androstene-3,6,17-triune, bicalutamide, posaconazole, fadrozole, dexmedetomidine, ravuconazole, or derivatives or combinations thereof. In some embodiments, the oxysterol inhibitor is anastrozole.

In some embodiments, the kits can further comprise instructional materials containing directions (i.e., protocols) for the practice of the methods of this invention (e.g., instructions for using the kit for treating Alzheimer's disease). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to Internet sites that provide such instructional materials.

### V. EXAMPLES

The following examples are offered to illustrate, but not to limit, the claimed invention.

### Example 1: Administration of a 27OHC Inhibitor to Reduce or Inhibit the Action of CYP27A1 on Cholesterol

This example describes an experimental protocol for testing the effect of an oxysterol inhibitor, anastrazole, on the cognitive function of mice fed a 0.5% cholesterol diet (based on all calories of diet), a diet that is associated with cognitive decline in the mice. Anastrozole is an FDA-approved aromatase inhibitor that has also been shown to inhibit the CYP27A1 enzyme (Mast et al., 2015, Mol. Pharmacol., 88:428-436). The CYP27A1 enzyme catalyzes cholesterol into an oxysterol called 27-hydroxycholesterol (27OHC). Studies have shown that CYP27A1 knockout mice fed a high cholesterol diet (0.5% cholesterol) showed no cognitive decline compared to wild type control mice fed the same diet (Heverin et al., 2015, Behav Brain Res., 278:356-359; data not shown).

In this study, mice are administered a standard lab chow supplemented with 0.5% cholesterol, anastrazole at a dosage of about 0.5 mg/day administered according to the method of Overk et al. (J. Neurosci Methods, 2011, 195:194-199), and 0.05% cholic acid. Mice diets are supplemented with 0.05% cholic acid because inhibition of CYP27A1 affects the production of bile acids and absorption of cholesterol and other lipids; supplementation with 0.05% cholic acid helps maintain normal levels of bile acid synthesis and absorption of cholesterol and lipid (Bavner et al., J. Lipid Res., 2010, 51:2722-30). Mice are divided into 2 groups (n=8 per group), fed either 0% or 0.5% cholesterol diets for 6 weeks. Cognitive function is measured using one or more models that measure cognitive function (e.g., Open Field (See, Stanford, J. Psychopharmacology, 2007, 21(2)134-5), Y-Maze and Novel Object Recognition tests (see, Webster et al., Frontiers in Genetics, 2014, 5(88):1-23)).

### Example 2: Effects of Anastrozole

A pilot study was carried out to assess the safety profile and cognitive impact of anastrozole in 12-week old male mice. Mice were fed anastrozole or placebo (vehicle alone) for a period of 8 weeks using a hydrogel as the means of administration (n=6 per group). Because of the poor palatability of anastrozole in its available form, it was administered through a hydration gel especially designed for medication delivery (LabGel-ClearH20 catalog # 71-02-1081; Portland, Me.), LabGel consists of purified water, sucralose, fruit and natural flavoring, hydrocolloids, potassium sorbate, sodium benzoate, and phosphoric acid. For each 236 mL dose of LabGel, 45 mg of anastrozole powder, 40 μL of red food coloring, and 1 mL of propylene glycol are thoroughly mixed and the resulting anastrozole is distributed into mouse cages at 3 grams LabGel/mouse/day in a custom--made container easily accessible to the mice. The final concentration of anastrozole in the LabGel is 0.1906 mg/mL and it is kept fresh by replacing it every 48 hours. In the experiment, mice consumed about 2 mL/mouse/day of LabGel equating to about 0.4 mg anastrozole/animal/day, which is consistent with the literature (Overk et al., J Neurosci Methods, 195: 194-9, 2011). Mice were monitored for health decline signs, and given an Appearance Score (Normal skin; skin tent on dorsum; hunched posture; eyes sunken in and severe skin tent; and failure to right self) and an Attitude Score (Normal; decreased activity; lethargy; non-responsiveness; and failure to flee when hand present in cage), as described (Bekkevold et al., J Am Assoc Lab Anim Sci, 52: 233-9, 2013). No signs of health decline based on these scores for any of these parameters were observed in vehicle or anastrozole-fed mice. Furthermore, weight gain progression was identical between both groups throughout the duration of the study and postmortem evaluation of major organs showed no signs of toxicity, an outcome that is otherwise expected based on exhaustive animal toxicology studies, listed in the National Library of Medicine Toxicology Data Network.

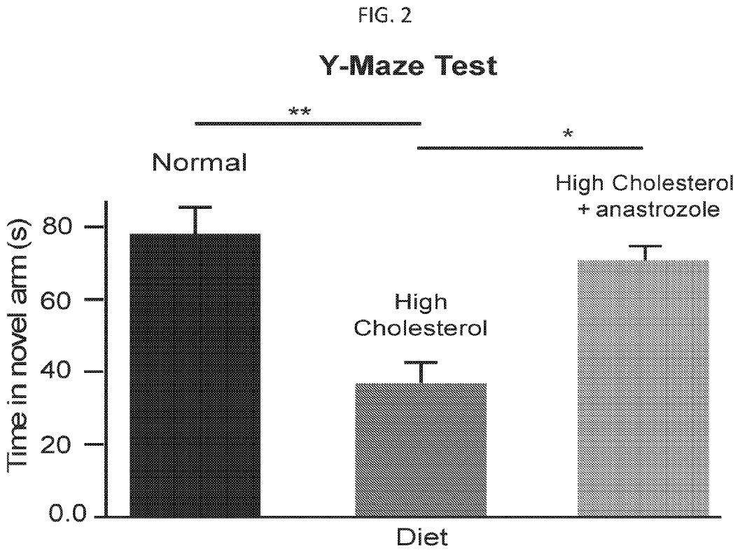

As can be seen in FIG. 2, after 8 weeks of treatment, anastrozole protects against cognitive loss in mice fed a high-cholesterol diet, such as standard chow containing 0.5% cholesterol, as measured by Y-maze test. 12-week old male mice on either a normal or high-cholesterol diet were tested on the same day with an interval of 2 hours between the training 15 min) and testing (5 min) phases. Data were analyzed by one-way ANOVA and Bonferroni post-hoc test. Error bars represent S.E.M. **p<0.01; *p<0.05.

This application incorporates by reference the contents of co-pending, unpublished PCT Application PCT/US2017/065367, filed Dec. 8, 2017, which claims priority to U.S. Provisional Patent Application No. 62/432,091, filed Dec. 9, 2016, the entire contents of both patent applications are incorporated herein by reference in their entirety, for all purposes.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

What is claimed is:

1. A method of delaying or reversing progression of Alzheimer's disease in a subject, the method comprising:
administering to a subject having Alzheimer's disease an oxysterol inhibitor.

2. (canceled)

3. (canceled)

4. (canceled)

5. (canceled)

6. The method of claim 1, wherein the oxysterol inhibitor is a 27-hydroxycholesterol (27OHC) inhibitor.

7. (canceled)

8. The method of claim 1, wherein the oxysterol inhibitor is an aromatase inhibitor.

9. The method of claim 1, wherein the oxysterol inhibitor is selected from the group consisting of anastrozole, exemestane, letrozole, vorozole, formestane, testolactone, aminoglutethimide, 1,4,6-androstatrien-3,17-dione, 4-androstene-3,6,17-trione, bicalutamide, posaconazole, fadrozole, dexmedetomidine, ravuconazole, and derivatives or combinations thereof.

10. The method of claim 1, wherein the oxysterol inhibitor is anastrozole.

11. The method of claim 1, wherein administration of the oxysterol inhibitor delays progression of mild Alzheimer's disease to moderate Alzheimer's disease.

12. The method of claim 1, wherein administration of the oxysterol inhibitor delays progression to severe Alzheimer's disease.

13. (canceled)

14. (canceled)

15. A method of delaying the onset of Alzheimer's disease in a subject having Mild Cognitive Impairment, the method comprising administering to the subject having Mild Cognitive Impairment an oxysterol inhibitor.

16. The method of claim 15, wherein the oxysterol inhibitor is a 27OHC inhibitor.

17. (canceled)

18. The method of claim 15, wherein the oxysterol inhibitor is an aromatase inhibitor.

19. The method of claim 15, wherein the oxysterol inhibitor is selected from the group consisting of anastrozole, exemestane, letrozole, vorozole, formestane, testolactone, aminoglutethimide, 1,4,6-androstatrien-3,17-dione, 4-androstene-3,6,17-trione, bicalutamide, posaconazole, fadrozole, dexmedetomidine, ravuconazole, and derivatives or combinations thereof.

20. The method of claim 15, wherein the oxysterol inhibitor is anastrozole.

21. The method of claim 15, wherein administration of the oxysterol inhibitor to the subject results in an increased level of expression of rhotekin 2 (RTKN2) in the subject.

22. A method of preventing or delaying the onset of Alzheimer's disease or Mild Cognitive Impairment in a subject who is at least 60 years old and/or has a cholesterol level of 200 mg/dL or above, the method comprising administering to the subject an oxysterol inhibitor.

23. The method of claim 22, wherein the oxysterol inhibitor is a 27OHC inhibitor.

24. (canceled)

25. The method of claim 22, wherein the oxysterol inhibitor is an aromatase inhibitor.

26. The method of claim 22, wherein the oxysterol inhibitor is selected from the group consisting of anastrozole, exemestane, letrozole, vorozole, formestane, testolactone, aminoglutethimide, 1,4,6-androstatrien-3,17-dione, 4-androstene-3,6,17-trione, bicalutamide, posaconazole, fadrozole, dexmedetomidine, ravuconazole, and derivatives or combinations thereof.

27. The method of claim 22, wherein the oxysterol inhibitor is anastrozole.

28. The method of claim 22, wherein administration of the oxysterol inhibitor to the subject results in an increased level of expression of rhotekin 2 (RTKN2) in the subject.
